(19) United States
(12) Patent Application Publication
(10) Pub. No.: US 2013/0158408 A1
(43) Pub. Date: Jun. 20, 2013

(54) ULTRASONIC DIAGNOSTIC DEVICE

(71) Applicant: GE Medical Systems Global Technology Company, LLC, Waukesha, WI (US)

(72) Inventors: Masaki Tsuda, Tokyo (JP); Shinichi Amemiya, Tokyo (JP)

(73) Assignee: GE MEDICAL SYSTEMS GLOBAL TECHNOLOGY COMPANY, LLC, Waukesha, WI (US)

(21) Appl. No.: 13/714,943

(22) Filed: Dec. 14, 2012

(30) Foreign Application Priority Data
Dec. 16, 2011 (JP) 2011-275133

(51) Int. Cl. A61B 8/00; A61B 8/08; A61B 8/14

(52) U.S. Cl. CPC A61B 8/54; A61B 8/4444; A61B 8/4254; A61B 8/14; A61B 8/462; A61B 8/5207; A61B 8/488
USPC 600/459

(57) ABSTRACT

An ultrasonic diagnostic device is provided. The ultrasonic diagnostic device includes an ultrasonic probe configured to send and receive ultrasonic waves to and from a test subject, a sensor provided in the ultrasonic probe and configured to detect a dynamic state of the ultrasonic probe, a dynamic analysis section configured to analyze the dynamic state of the ultrasonic probe based on a detection signal of the sensor, and a control section configured to perform a control operation of the ultrasonic diagnostic device corresponding to the dynamic state of the ultrasonic probe as identified from the analysis by the dynamic analysis section.

### CROSS REFERENCE TO RELATED APPLICATIONS

This application claims the benefit of Japanese Patent Application No. 2011-275133 filed Dec. 16, 2011, which is hereby incorporated by reference in its entirety.

### BACKGROUND OF THE INVENTION

The present invention relates to an ultrasonic diagnostic device which can be operated by an ultrasonic probe.

In the ultrasonic diagnostic device, an operation section is provided on the device main body and input for operating the ultrasonic diagnostic device is made on the operation section to control the ultrasonic diagnostic device.

However, for an operator having an ultrasonic probe, it may be more convenient to do input work for operation on the ultrasonic probe than do input work for operation on the operation section. For the above reason, for example, JP-A No. 2002-301075 discloses an ultrasonic probe with buttons for input for operation.

On the operation section, input for operation is made for a wide variety of items. However, it is impossible to provide many buttons on an ultrasonic probe and items for which operation can be done by buttons are limited.

In addition, the ultrasonic probe may be immersed in a liquid for sterilization after its use, so a button must be provided in a watertight manner. Furthermore, it must be designed so that the button is located in a way not to interfere with scanning operation and in a way to ensure operating ease.

### BRIEF DESCRIPTION OF THE INVENTION

The systems and methods described herein include an ultrasonic diagnostic device which can be operated by an ultrasonic probe without a button. Specifically, in one aspect, an ultrasonic diagnostic device is provided. The ultrasonic diagnostic device includes an ultrasonic probe which sends and receives ultrasonic waves to and from a test subject, a sensor provided in the ultrasonic probe for detecting the dynamic state of the ultrasonic probe, a dynamic analysis section which analyzes the dynamic state of the ultrasonic probe based on a detection signal of the sensor, and a control section which performs control of the ultrasonic diagnostic device corresponding to the dynamic state of the ultrasonic probe as identified based on analysis by the dynamic analysis section.

In another aspect, an ultrasonic diagnostic device is provided. The ultrasonic diagnostic device includes an ultrasonic probe which sends and receives ultrasonic waves to and from a test subject, a sound sensor provided in the ultrasonic probe, an operation sound identifying section which identifies the sound of operation for carrying out operation by the ultrasonic probe, based on a detection signal of the sound sensor, and a control section which performs control of the ultrasonic diagnostic device corresponding to the operation sound identified by the operation sound identifying section.

According to the above aspect, the dynamic state of the ultrasonic probe is analyzed by the dynamic analysis section and control which corresponds to the dynamic state of the ultrasonic probe is performed by the control section. Therefore, the ultrasonic diagnostic device can be controlled based on the dynamic state of the ultrasonic probe. Consequently, operation by the ultrasonic probe can be done without a button provided on the ultrasonic probe.

According to the above other aspect, the sound of operation for carrying out operation by the ultrasonic probe is identified by the operation sound identifying section and control of the ultrasonic diagnostic device corresponding to the operation sound identified by the operation sound identifying section is performed. Therefore, the ultrasonic diagnostic device can be controlled based on the sound of operation for carrying out operation by the ultrasonic probe. Consequently, operation by the ultrasonic probe can be done without a button provided on the ultrasonic probe.

### BRIEF DESCRIPTION OF THE DRAWINGS

FIG. 1 is a block diagram showing an example of the general configuration of an ultrasonic diagnostic device in a first embodiment.

FIG. 2 is a block diagram showing an example of the general configuration of the ultrasonic diagnostic device in a variation of the first embodiment.

FIG. 3 is a block diagram showing an example of the general configuration of an ultrasonic diagnostic device in a second embodiment.

FIG. 4 is a block diagram showing an example of the general configuration of the ultrasonic diagnostic device in a variation of the second embodiment.

### DETAILED DESCRIPTION OF THE INVENTION

Next, exemplary embodiments will be described.

### First Embodiment

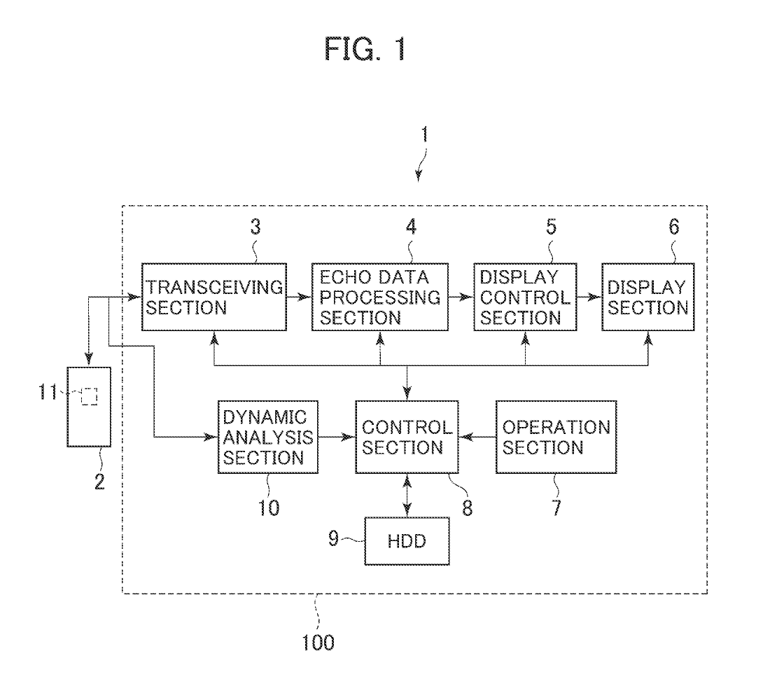

First, the first embodiment will be described referring to FIG. 1. An ultrasonic diagnostic device 1 shown in FIG. 1 includes an ultrasonic probe 2, a transceiving section 3, an echo data processing section 4, a display control section 5, a display section 6, an operation section 7, a control section 8, a HDD (Hard Disk Drive) 9, and a dynamic analysis section 10. The transceiving section 3, the echo data processing section 4, the display control section 5, the display section 6, the operation section 7, the control section 8, the HDD 9, and the dynamic analysis section 10 are provided in the device main body 100. The ultrasonic probe 2 is connected with the device main body 100.

The ultrasonic probe 2 is structured to have a plurality of ultrasonic oscillators (not shown) arranged in an array pattern and sends ultrasonic waves to a test subject through the ultrasonic oscillators and receives their echo signals. The ultrasonic probe 2 is an example of an embodiment of the ultrasonic probe.

The ultrasonic probe 2 is provided with an acceleration sensor 11. This acceleration sensor 11 is an example of an embodiment of the sensor. The acceleration sensor 11 is, for example, MEMS (Micro Electro Mechanical Systems) sensor such as a capacitance type, piezoresistance type, or heat detection type. In the exemplary embodiment, the acceleration sensor 11 is a three-axis acceleration sensor. However, it is not limited thereto.

A detection signal in the acceleration sensor 11 is inputted into the dynamic analysis section 10. The dynamic analysis section 10 analyzes the dynamic state of the ultrasonic probe 2 based on the detection signal inputted from the acceleration sensor 11. The dynamic state here means movement or vibration of the ultrasonic probe 2. It will be described in detail later. The dynamic analysis section 10 is an example of an embodiment of the dynamic analysis section.

The transceiving section 3 supplies an electric signal for sending ultrasonic waves from the ultrasonic probe 2 under a prescribed scanning condition, based on a control signal from the control section 8. Also the transceiving section 3 performs signal processing, such as A/D conversion and phasing addition, of an echo signal received by the ultrasonic probe 2 and outputs the echo data after signal processing to the echo data processing section 4.

The echo data processing section 4 processes the echo data outputted from the transceiving section 3 for generating an ultrasonic image. For example, the echo data processing section 4 generates B-mode data by B-mode processing such as logarithmic compression and envelop demodulation. Also the echo data processing section 4 may not only perform B-mode processing but also generate doppler data by doppler processing such as quadrature detection and filtering.

The display control section 5 scans and converts the data inputted from the echo data processing section 4 by a scan converter to generate ultrasonic image data. For example, the display control section 5 scans and converts the B-mode data to generate B-mode image data. Also the display control section 5 may generate color doppler image data by scanning and converting the doppler data, as well as B-mode image data.

The display control section 5 makes the display section 6 display a B-mode image based on the B-mode image data and a color doppler image based on the color doppler image data.

The display section 6 is an LCD (Liquid Crystal Display) or CRT (Cathode Ray Tube). The operation section 7 is structured to include a keyboard and pointing device (not shown) for an operator to enter an instruction or data.

The control section 8 is structured to have a CPU (Central Processing Unit) though it is not shown in the figure. The control section 8 reads a control program stored in the HDD 9 and controls operation of each section of the ultrasonic diagnostic device 1 to let it perform a prescribed function.

The control section 8 controls operation of each section of the ultrasonic diagnostic device 1 according to input on the operation section 7. Also the control section 8 controls operation of each section of the ultrasonic diagnostic device 1 corresponding to the dynamic state of the ultrasonic probe 2 as identified based on analysis by the dynamic analysis section 10. The control section 8 is an example of an embodiment of the control section.

The HDD 9 stores information Inf on operation control which corresponds to the dynamic state of the ultrasonic probe 2. The HDD 9 is an example of the storage section. The control section 8 controls operation of each section of the ultrasonic diagnostic device 1 corresponding to the dynamic state of the ultrasonic probe 2, based on the information Inf stored in the HDD 9.

Next, the function of the ultrasonic diagnostic device 1 in this example will be described. In order to display an ultrasonic image on the ultrasonic diagnostic device 1, an echo signal is obtained by the ultrasonic probe 2 sending and receiving ultrasonic waves to and from the test subject. Based on this echo signal, the echo data processing section 4 performs, for example, B-mode processing to generate B-mode data and the display control section 5 generates B-mode image data to let the display section 6 display a B-mode image in real time.

While a B-mode image is displayed in real time, if an operator wants to freeze the B-mode image, for example, the operator taps the surface of the ultrasonic probe 2 with a finger once. The vibration due to this tap is detected by the acceleration sensor 11 and the detection signal is inputted into the dynamic analysis section 10. The dynamic analysis section 10 analyzes the dynamic state of the ultrasonic probe 2 based on the detection signal and outputs the analysis result to the control section 8. In this case, the dynamic analysis section 10 outputs, to the control section 8, the analysis result that the ultrasonic probe 2 has been tapped once.

Here, the HDD 9 stores ultrasonic image freezing operation as control corresponding to a single tap in the information Inf on the operation control which corresponds to the dynamic state of the ultrasonic probe 2. Therefore, the control section 8 freezes the B-mode image as control corresponding to a single tap based on the operation control information Inf.

Other types of control may be stored in the operation control information Inf. For example, the dynamic state of the ultrasonic probe 2 which corresponds to unfreezing or B-mode or color doppler mode switching control may be stored as the operation control information Inf. For example, the operation control information Inf may be stored so that if the ultrasonic probe 2 is tapped twice successively, unfreezing operation should be done. Also the operation control information Inf may be stored so that if the ultrasonic probe 2 is moved in a way to draw the letter “B” in three-dimensional space, B-mode imaging control should be performed and if the ultrasonic probe 2 is moved in a way to draw the letter “C”, color doppler mode imaging control should be performed.

According to the ultrasonic diagnostic device 1 in this example, operation can be carried out by tapping or moving the ultrasonic probe 2, so it is convenient for an operator. In addition, the ultrasonic probe 2 need not be provided with a button for operation by the ultrasonic probe 2.

Furthermore, since control operation which corresponds to the dynamic state of the ultrasonic probe 2 is to be stored, the number of times of operation control can be larger than when an operation button is provided on the ultrasonic probe 2. In addition, there is no need to devise the structure for the provision of a button and its position.

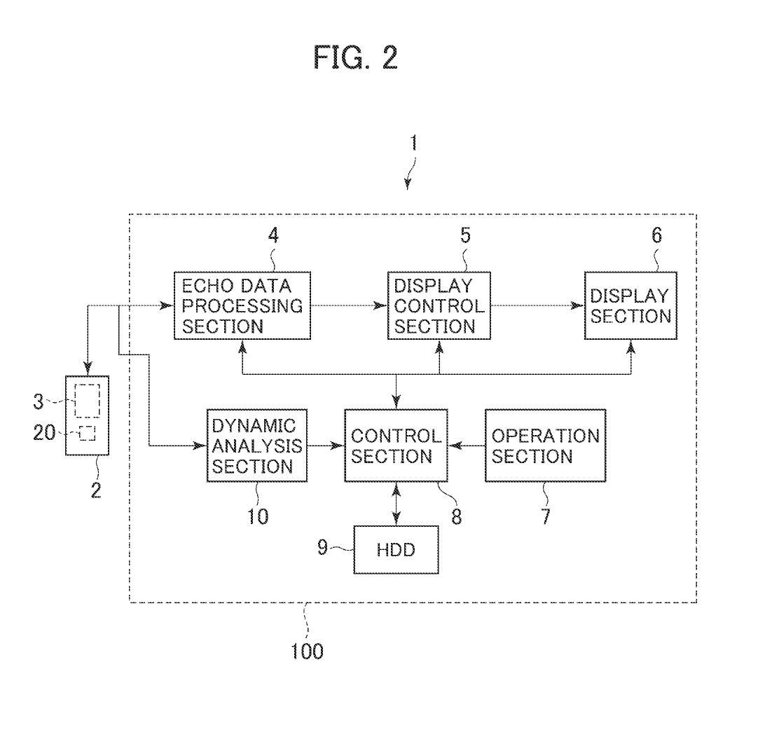

Next, a variation of the first embodiment will be described referring to FIG. 2. In this variation, the transceiving section 3 may be provided in the ultrasonic probe 2. In this case as well, a control signal from the control section 8 is inputted into the transceiving section 3.

### Second Embodiment

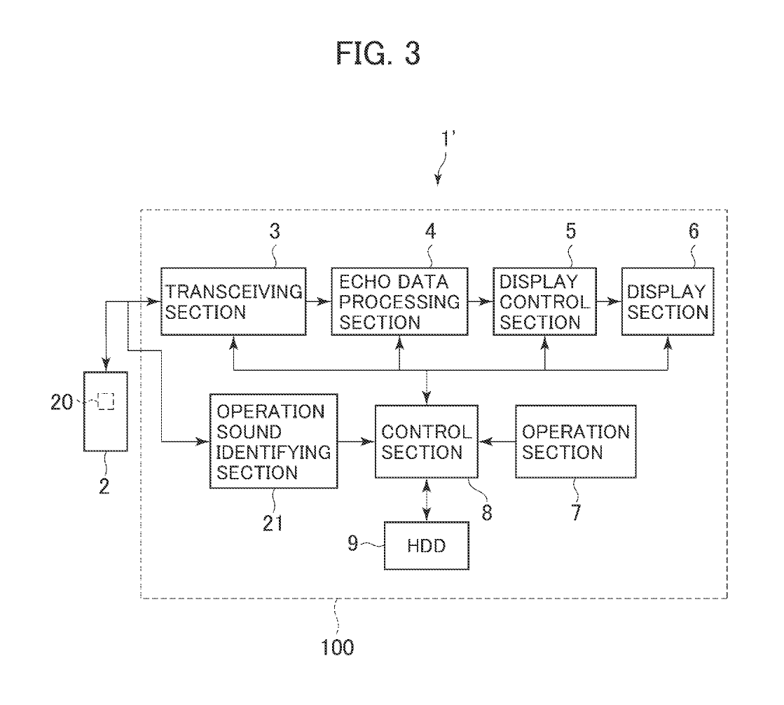

Next, the second embodiment will be described referring to FIG. 3. Description of the same matters as in the first embodiment is omitted.

In the ultrasonic diagnostic device 1′ in this example, a sound sensor 20 is provided in the ultrasonic probe 2. Also an operation sound identifying section 21 is provided in the device main body 100 in place of the dynamic analysis section 10.

The sound sensor 20 is a microphone. This sound sensor 20 is an example of an embodiment of the sound sensor. A detection signal of the sound sensor 20 is inputted into the operation sound identifying section 21. This operation sound identifying section 21 identifies the sound of operation for carrying out operation by the ultrasonic probe 2, based on the detection signal of the sound sensor 20. The operation sound identifying section 21 is an example of an embodiment of the operation sound identifying section.

In this embodiment, operation sound is tapping sound generated by an operator tapping the surface of the ultrasonic probe 2 with a finger. The HDD 9 stores information Inf on operation control corresponding to tap sound. For example, the operation control information Inf may be stored so that if sound is made by tapping once, freezing operation should be done and if sound is made by tapping twice, unfreezing operation should be done. In this case, the operation sound identifying section 21 identifies the type of tap sound, namely whether it is single-tap sound or double-tap sound.

The control section 8 performs operation control of the ultrasonic diagnostic device 1′ which corresponds to the operation sound identified by the operation sound identifying section 21. For example, if the tap sound is single-tap sound, the control section 8 freezes the ultrasonic image displayed on the display section 6. If the tap sound is double-tap sound, the control section 8 unfreezes the ultrasonic image displayed on the display section 6.

According to the ultrasonic diagnostic device 1′ in this example, operation can be done according to the sound of tapping the ultrasonic probe 2, the same advantage as in the first embodiment can be obtained.

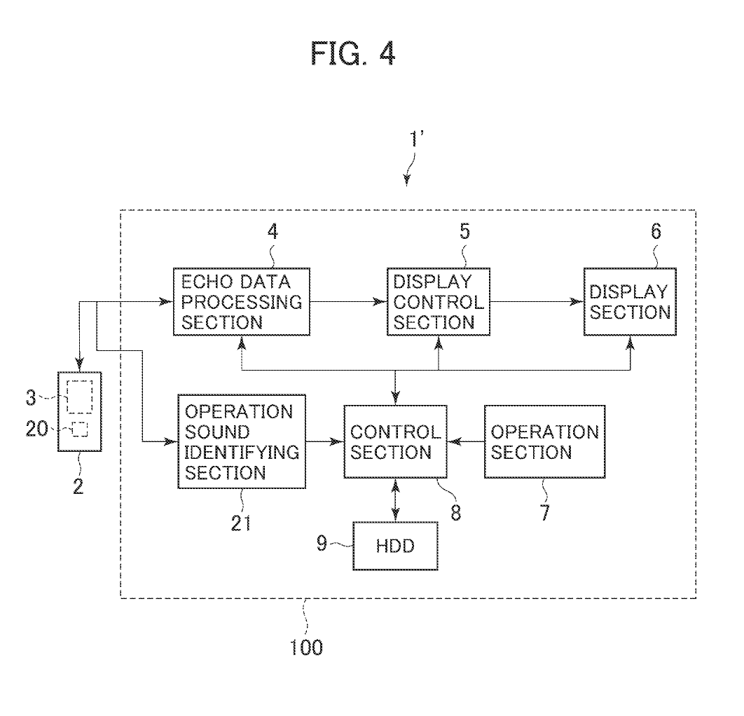

Next, a variation of the second embodiment will be described referring to FIG. 4. In this variation, like the variation of the first embodiment, the transceiving section 3 may be provided in the ultrasonic probe 2. In this case as well, a control signal from the control section 8 is inputted into the transceiving section 3.

Exemplary embodiments have been described but it is needless to say that the systems and methods described herein may be modified and embodied in various ways without changing the gist of the invention. For example, although the above embodiments have been described on the assumption that freezing, unfreezing or imaging mode switching is done according to the dynamic state of the ultrasonic probe 2 or the sound of tapping the ultrasonic probe 2, control other than these kinds of control may be performed.

In the claims:

1. An ultrasonic diagnostic device comprising:
an ultrasonic probe configured to send and receive ultrasonic waves to and from a test subject;
a sensor provided in the ultrasonic probe and configured to detect a dynamic state of the ultrasonic probe;
a dynamic analysis section configured to analyze the dynamic state of the ultrasonic probe based on a detection signal of the sensor; and
a control section configured to perform a control operation of the ultrasonic diagnostic device corresponding to the dynamic state of the ultrasonic probe as identified from the analysis by the dynamic analysis section.

2. The ultrasonic diagnostic device according to claim 1, wherein the sensor is a sensor configured to detect at least one of vibration and movement.

3. The ultrasonic diagnostic device according to claim 1, wherein the sensor is an acceleration sensor.

4. The ultrasonic diagnostic device according to claim 2, wherein the sensor is an acceleration sensor.

5. The ultrasonic diagnostic device according to claim 1, further comprising a storage section configured to store the control operation corresponding to the dynamic state of the ultrasonic probe.

6. The ultrasonic diagnostic device according to claim 2, further comprising a storage section configured to store the control operation corresponding to the dynamic state of the ultrasonic probe.

7. The ultrasonic diagnostic device according to claim 3, further comprising a storage section configured to store the control operation corresponding to the dynamic state of the ultrasonic probe.

8. The ultrasonic diagnostic device according to claims 4, further comprising a storage section configured to store the control operation corresponding to the dynamic state of the ultrasonic probe.

9. An ultrasonic diagnostic device comprising:
an ultrasonic probe configured to send and receive ultrasonic waves to and from a test subject;
a sound sensor provided in the ultrasonic probe;
an operation sound identifying section configured to identify a sound of operation based on a detection signal of the sound sensor; and
a control section configured to perform a control operation of the ultrasonic diagnostic device corresponding to the sound of operation identified by the operation sound identifying section.

10. The ultrasonic diagnostic device according to claim 9, further comprising a storage section configured to store the control operation corresponding to the sound of operation.

11. The ultrasonic diagnostic device according to claim 9, wherein the operation sound identifying section is configured to identify a sound of the ultrasonic probe being tapped by an operator.

12. The ultrasonic diagnostic device according to claim 10, wherein the operation sound identifying section is configured to identify a sound of the ultrasonic probe being tapped by an operator.

13. A method of controlling an ultrasonic diagnostic device, the method comprising:
sending and receiving ultrasonic waves to and from a test subject using an ultrasonic probe;
detecting a dynamic state of the ultrasonic probe using a sensor provided in the ultrasonic probe;
analyzing the dynamic state of the ultrasonic probe based on a detection signal of the sensor; and
performing, using a control section, a control operation of the ultrasonic diagnostic device corresponding to the dynamic state of ultrasonic probe as identified from the analysis of the dynamic state.

14. The method according to claim 13, wherein analyzing the dynamic state comprises analyzing the dynamic state using a dynamic analysis section provided in the ultrasonic diagnostic device.

15. The method according to claim 13, wherein detecting a dynamic state comprises detecting at least one of vibration and movement of the ultrasonic probe.

16. The method accordingly to claim 13, wherein detecting a dynamic state comprises detecting a dynamic state using an acceleration sensor.

17. The method accordingly to claim 15, wherein detecting a dynamic state comprises detecting a dynamic state using an acceleration sensor.

18. The method accordingly to claim 13, further comprising storing the control operation corresponding to the dynamic state of the ultrasonic probe in a storage section provided in the ultrasonic diagnostic device.

19. The method accordingly to claim 15, further comprising storing the control operation corresponding to the dynamic state of the ultrasonic probe in a storage section provided in the ultrasonic diagnostic device.

20. The method accordingly to claim 16, further comprising storing the control operation corresponding to the dynamic state of the ultrasonic probe in a storage section provided in the ultrasonic diagnostic device.
